(19) United States
(12) Patent Application Publication
(10) Pub. No.: US 2021/0077068 A1
(43) Pub. Date: Mar. 18, 2021

(54) SYSTEMS AND METHODS FOR AUTOMATED ULTRASOUND IMAGE LABELING AND QUALITY GRADING

(71) Applicant: EchoNous, Inc., Redmond, WA (US)

(72) Inventors: Allen Lu, Seattle, WA (US); Matthew Cook, Woodinville, WA (US); Babajide Ayinde, Redmond, WA (US); Nikolaos Pagoulatos, Kirkland, WA (US); Ramachandra Pailoor, Woodinville, WA (US)

(21) Appl. No.: 17/018,837

(22) Filed: Sep. 11, 2020

Related U.S. Application Data
(60) Provisional application No. 62/899,554, filed on Sep. 12, 2019.

(51) Int. Cl. A61B 8/00; A61B 8/08; G06K 9/78; G06T 7/00; G06N 3/04; G06N 3/08

(52) U.S. Cl. CPC A61B 8/42; A61B 8/463; A61B 8/085; G06K 9/78; G06T 7/0012; G06T 2207/20084; G06N 3/08; G06K 2209/05; G06T 2207/10132; G06T 2207/30168; G06T 2207/20081; G06N 3/04

(57) ABSTRACT

Automated ultrasound image labeling and quality grading systems and methods are provided. An ultrasound system includes an ultrasound imaging device configured to acquire ultrasound images of a patient. An anatomical structure recognition and labeling module receives the acquired ultrasound images from the ultrasound imaging device, and automatically recognizes anatomical structures in the received ultrasound images. The anatomical structure recognition and labeling module automatically labels the anatomical structures in the images with information that identifies the anatomical structures. The acquired ultrasound images and the labeled anatomical structures are displayed on a display of the ultrasound imaging device.

### BACKGROUND

### Technical Field

This disclosure generally relates to ultrasound imaging systems and methods and, more particularly, to artificial intelligence based networks for ultrasound imaging and evaluation of ultrasound images, and systems and methods for automatically recognizing and labeling anatomical structures in acquired ultrasound images and for grading an image quality of acquired ultrasound images.

### Description of the Related Art

Ultrasound imaging is typically performed in a clinical setting, by trained ultrasound experts. For diagnostic ultrasound imaging, particular views of an organ or other tissue or body feature (such as fluids, bones, joints or the like) are clinically significant. Such views may be prescribed by clinical standards as views that should be captured by the ultrasound technician, depending on the target organ, diagnostic purpose or the like.

The image quality of acquired ultrasound images varies depending on a variety of factors, including, for example, the positioning of the probe, the imaging parameters (e.g., depth, gain, etc.), and so on. For clinical use (e.g., for diagnosis), ultrasound images generally should have suitable image quality. Clinicians generally require significant training in order to assess the diagnostic quality of ultrasound images. Such images can be obtained in real time during image acquisition or they can be previously acquired. In both cases, clinicians need to understand the level of diagnostic quality of the ultrasound images. Similarly, in training and educational settings, expert ultrasound users are required to grade the diagnostic quality of images acquired by students and novice users, and this is very time consuming for the ultrasound experts.

Moreover, significant training is generally required for clinicians to be able to recognize the anatomical structures present in an ultrasound image. This is particularly challenging during real time ultrasound image acquisition during which the ultrasound images change continuously in real time as the position and orientation of the probe moves with respect to the organ of interest.

While conventional ultrasound imaging systems may be suitable for most patients in a hospital or similar clinical setting, such systems require significant training to operate and to adequately capture clinically desirable views. This adds to the overall cost of such ultrasound imaging and further limits the availability of ultrasound imaging to patients, as only well-trained professionals can properly operate conventional ultrasound imaging devices.

### BRIEF SUMMARY

The present disclosure provides systems and methods that facilitate automated ultrasound image labeling and automated ultrasound image quality grading. In particular, the systems and methods provided herein are operable to recognize anatomical structures within acquired ultrasound images and to label the recognized anatomical structures with information which identifies the anatomical structures. The labels may be displayed on a display device along with the acquired ultrasound image, for example, the labels may be superimposed on the ultrasound image at positions or regions which correspond to the positions or regions of the recognized anatomical structures. Moreover, the systems and methods provided herein are operable to automatically grade an image quality of the acquired ultrasound images, and the grade may be displayed or otherwise provided to a user, and in some embodiments, the grade may be utilized to help guide the user toward acquisition of higher quality images, such as to ultrasound images representing a clinically-desirable view of an organ or other body feature.

In various embodiments, machine learning techniques are utilized to automatically grade the diagnostic quality of ultrasound images, which solves the problems of: (i) new and novice ultrasound users not knowing the diagnostic quality of their images during acquisition, and (ii) expert instructors having to spend significant amounts of time to grade the diagnostic quality of images acquired by new/novice users. Embodiments provided herein apply advanced machine learning approaches to automatically grade the diagnostic quality of ultrasound images, and the grade may be based on well-established image quality scales or criteria provided by the clinical community.

In various embodiments, the problem of correctly identifying anatomical structures in ultrasound images is solved by applying machine learning algorithms to automatically perform the recognition and labeling of such anatomical structures, either in real-time during acquisition or post acquisition, or both. Advanced machine learning approaches are applied in various embodiments to not only recognize key anatomical structures in the image but to also localize them, i.e. to determine the position in the image where each anatomical structure is present.

In at least one embodiment, an ultrasound system is provided that includes an ultrasound imaging device and anatomical structure recognition and labeling circuitry. The ultrasound imaging device acquires ultrasound images of a patient. The anatomical structure recognition and labeling circuitry receives the acquired ultrasound images, automatically recognizes one or more anatomical structures in the received ultrasound images, and automatically labels the one or more anatomical structures in the images with information that identifies the one or more anatomical structures. The ultrasound imaging devices includes a display that displays the acquired ultrasound images and the labeled one or more anatomical structures.

In at least one embodiment, a method is provided that includes: receiving, by anatomical structure recognition and labeling circuitry, ultrasound images acquired by an ultrasound imaging device; automatically recognizing, by the anatomical structure recognition and labeling circuitry, one or more anatomical structures in the received ultrasound images; automatically labeling, by the anatomical structure recognition and labeling circuitry, the one or more anatomical structures in the acquired ultrasound images with information that identifies the one or more anatomical structures; and displaying the acquired ultrasound images and the labeled one or more anatomical structures.

In at least one embodiment, an ultrasound system includes ultrasound image grading circuitry configured to receive acquired ultrasound images from an ultrasound imaging device, and automatically grade an image quality of the received ultrasound images. A display is included that is configured to concurrently display the acquired ultrasound images and an indication of the image quality grade.

### BRIEF DESCRIPTION OF THE SEVERAL VIEWS OF THE DRAWINGS

FIG. 1 is a block diagram illustrating an automated ultrasound image labeling and quality grading system, in accordance with one or more embodiments of the disclosure;

FIG. 2 is a block diagram illustrating training of the machine learning circuitry of the system shown in FIG. 1, in accordance with one or more embodiments of the disclosure;

FIG. 3 is a block diagram illustrating a neural network, which may be implemented by the machine learning circuitry, in accordance with one or more embodiments of the disclosure;

FIG. 4 is a schematic illustration of an ultrasound imaging device, in accordance with one or more embodiments of the disclosure;

FIG. 5 is a view illustrating an automatically labeled ultrasound image, in accordance with one or more embodiments of the disclosure; and

FIGS. 6A and 6B are views illustrating ultrasound images including grades indicating a quality of the ultrasound images, in accordance with one or more embodiments.

### DETAILED DESCRIPTION

The present disclosure provides several embodiments of systems and methods for automatic ultrasound image labeling and quality grading, as well as systems and methods for ultrasound image recognition. The systems and methods provided herein may be particularly useful for ultrasound imaging performed by novice ultrasound technicians and/or for ultrasound imaging utilizing a handheld or mobile ultrasound imaging device which may be deployed in a non-traditional clinical setting. Utilizing artificial intelligence approaches, the systems and methods provided herein are capable of automatically recognizing and labeling anatomical structures within acquired ultrasound images. The labels may be displayed with the ultrasound image, e.g., superimposed onto the corresponding anatomical structures in the image. Artificial intelligence approaches are also utilized in the systems and methods provided herein to automatically determine an image quality grade for acquired ultrasound images, and in some embodiments, the determined image quality grade may be utilized to guide the user toward acquisition of a particular ultrasound image, such as a particular clinically desirable or standard view.

In various embodiments, the systems and methods provided herein may further be utilized to determine whether acquired ultrasound images accurately depict or represent a desired view of a patient's organ or other tissue, feature or region of interest in a patient.

The systems and methods provided herein may provide feedback to a user, for example, to indicate a determined image quality of the acquired ultrasound images, as well as to indicate whether or not a desired view of a patient's organ or other tissue or feature has been captured. In some embodiments, ultrasound images are displayed along with labels which are applied to recognized anatomical structures in the ultrasound images.

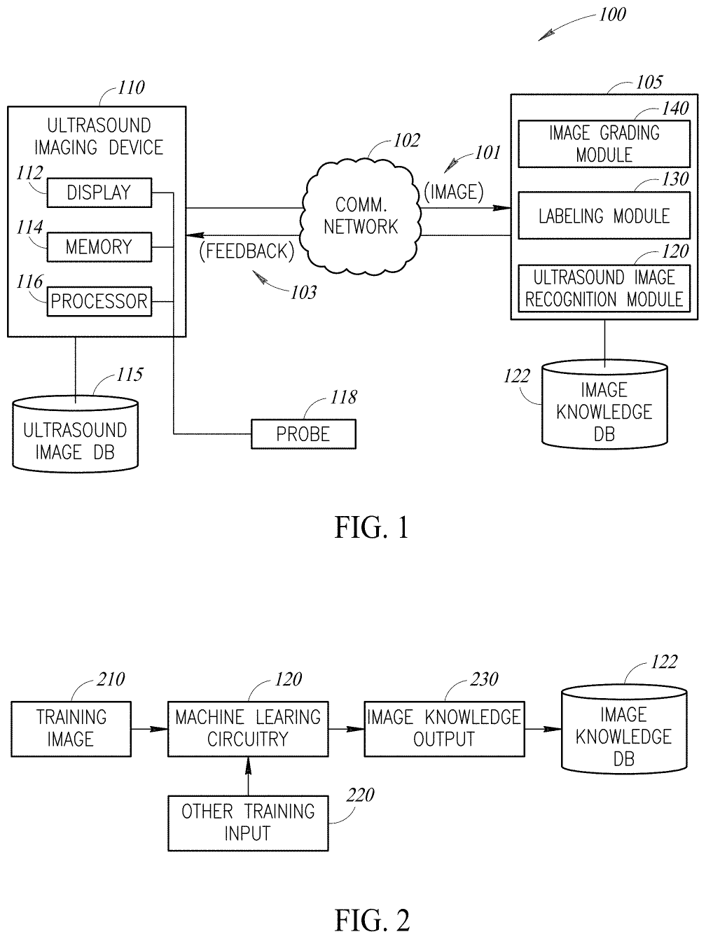

FIG. 1 illustrates a block diagram of an automated ultrasound image labeling and quality grading system 100 (which may be referred to herein as ultrasound system 100), in accordance with embodiments of the present disclosure. As shown in FIG. 1, the ultrasound system 100 includes an ultrasound imaging device 110, a communications network 102, machine learning circuitry 105, and an image knowledge database 122. Each of these may be incorporated into a single ultrasound device, such as a hand-held or portable device, or may constitute multiple devices operatively linked or linkable to one another. As will be described in further detail herein, the machine learning circuitry 105 may include an ultrasound image recognition module 120, an anatomical structure recognition and labeling module 130, and an ultrasound image grading module 140, each of which may include programmed and/or hardwired circuitry configured to perform the functions or actions of the respective modules as described herein.

The ultrasound imaging device 110 is any ultrasound device operable to acquire ultrasound images of a patient, and may be, in at least some embodiments for example, a handheld ultrasound imaging device. The ultrasound imaging device 110 may include a display 112, memory 114, and one or more processors 116. The ultrasound imaging device 110 is operatively coupled to an ultrasound probe 118.

The memory 114 may be or include any computer-readable storage medium, including, for example, read-only memory (ROM), random access memory (RAM), flash memory, hard disk drive, optical storage device, magnetic storage device, electrically erasable programmable read-only memory (EEPROM), organic storage media, or the like.

The processor 116 may be any computer processor operable to execute instructions (e.g., stored in memory 114) to perform the functions of the ultrasound imaging device 110 as described herein.

The ultrasound probe 118 is driven by the ultrasound imaging device 110 to transmit signals toward a target region in a patient, and to receive echo signals returning from the target region in response to the transmitted signals. In operation, a user of the ultrasound device 110 may hold the probe 118 against a patient's body at a position and angle to acquire a desired ultrasound image. The signals received by the probe (i.e., the echo signals) are communicated to the ultrasound imaging device 110 and may form, or be processed to form, an ultrasound image of the target region of the patient. Further, the ultrasound images may be provided to the display 112, which may display the ultrasound images and/or any other relevant information to the user.

The ultrasound images thus acquired by the ultrasound imaging device 110 may be provided to the machine learning circuitry 105 via a communications network 102. Ultrasound images from the ultrasound imaging device 110 are provided to the machine learning circuitry 105, as shown by reference numeral 101. Communications network 102 may utilize one or more protocols to communicate via one or more physical networks, including local area networks, wireless networks, dedicated lines, intranets, the Internet, and the like.

In one or more embodiments, the machine learning circuitry 105 (including, for example, the ultrasound image recognition module 120, the anatomical structure recognition and labeling module 130, and the ultrasound image grading module 140) may be provided within the ultrasound imaging device 110, or a local copy of the machine learning circuitry 105 and/or ultrasound image knowledge stored in the image knowledge database 122 may be contained within the ultrasound imaging device 110, with the ultrasound imaging device 110 having access to a remotely located (e.g., stored on one or more server computers, or in the “cloud”) machine learning circuitry 105.

The machine learning circuitry 105 may be or include any electrical circuitry configured to perform the ultrasound image recognition, image labeling, and image grading techniques described herein. In some embodiments, the machine learning circuitry 105 may include or be executed by a computer processor, a microprocessor, a microcontroller, or the like, configured to perform the various functions and operations described herein with respect to the machine learning circuitry 105. For example, the machine learning circuitry 105 may be executed by a computer processor selectively activated or reconfigured by a stored computer program, or may be a specially constructed computing platform for carrying out the features and operations described herein. In some embodiments, the machine learning circuitry 105 may be configured to execute software instructions stored in any computer-readable storage medium, including, for example, read-only memory (ROM), random access memory (RAM), flash memory, hard disk drive, optical storage device, magnetic storage device, electrically erasable programmable read-only memory (EEPROM), organic storage media, or the like.

The machine learning circuitry 105 receives the ultrasound images acquired from the ultrasound imaging device 110, and automatically determines an image quality grade for each of the received ultrasound images, and automatically labels one or more anatomical structures in the received ultrasound images. For example, in some embodiments, the anatomical structure recognition and labeling module 130 (which may be included as part of the machine learning circuitry 105) automatically recognizes anatomical structures in the ultrasound images, and automatically associates labels with the recognized anatomical structures. In some embodiments, the labels associated with the recognized anatomical structures are displayed (e.g., on the display 112) superimposed on or embedded within the ultrasound image in a region at which the corresponding anatomical structures are displayed.

In some embodiments, the ultrasound image grading module 140 (which may be included as part of the machine learning circuitry 105) automatically determines an image quality grade for each of the received ultrasound images.

In some embodiments, the ultrasound image recognition module 120 (which may be included as part of the machine learning circuitry 105) automatically determines whether one or more of the received ultrasound images represents a clinically desirable view of an organ or other aspect, region or feature of the patient.

Each of the ultrasound image recognition module 120, the anatomical structure recognition and labeling module 130, and the ultrasound image grading module 140 may be implemented by a computationally intelligent system that employs artificial intelligence, drawing from an image knowledge database 122, to perform the functions of these modules as described herein (e.g., determining whether received ultrasound images represent a clinically desirable view, recognizing and labeling anatomical structures in the ultrasound images, and determining an image quality grade for the ultrasound images). Some or all of the functions of the ultrasound image recognition module 120, the anatomical structure recognition and labeling module 130, and the ultrasound image grading module 140 described herein may be performed automatically by the ultrasound image recognition module 120, the anatomical structure recognition and labeling module 130, and the ultrasound image grading module 140, for example, in response to receiving the acquired ultrasound images.

“Artificial intelligence” is used herein to broadly describe any computationally intelligent systems and methods that can learn knowledge (e.g., based on training data), and use such learned knowledge to adapt its approaches for solving one or more problems. Artificially intelligent machines may employ, for example, neural network, deep learning, convolutional neural network, and Bayesian program learning techniques to solve problems such as image recognition, anatomical structure recognition and labeling, and image quality grading. Further, artificial intelligence may include any one or combination of the following computational techniques: constraint program, fuzzy logic, classification, conventional artificial intelligence, symbolic manipulation, fuzzy set theory, evolutionary computation, cybernetics, data mining, approximate reasoning, derivative-free optimization, decision trees, and/or soft computing. Employing one or more computationally intelligent techniques, the machine learning circuitry 105 (e.g., including the ultrasound image recognition module 120, the anatomical structure recognition and labeling module 130, and the ultrasound image grading module 140) may learn to adapt to an unknown and/or changing environment for better performance.

The image knowledge database 122 may include a variety of information facilitating image analysis, with respect to received ultrasound images, by the ultrasound image recognition module 120, the anatomical structure recognition and labeling module 130, and the ultrasound image grading module 140.

In some embodiments, the image knowledge database 122 may contain information relating to various image views of various organs. For example, the image knowledge database 122 may include information associated with clinically standard or desirable views of a heart. The clinically standard views of a heart may include, for example, suprasternal, subcostal, short- and long-axis parasternal, 2-chamber apical, 3-chamber apical, 4-chamber apical and 5-chamber apical views. Additionally, the information associated with clinically standard views may be information associated with a three-dimensional view, a two-dimensional cross section view and/or a set of two-dimensional cross section views.

The image knowledge database 122 may be stored in any computer-readable storage medium accessible by the machine learning circuitry 105, including, for example, any of the ultrasound image recognition module 120, the anatomical structure recognition and labeling module 130, and the ultrasound image grading module 140.

FIG. 2 is a block diagram illustrating training of the machine learning circuitry 105, in accordance with one or more embodiments. Training of the machine learning circuitry 105 may include, in various embodiments, separate or concurrent training of each of the ultrasound image recognition module 120, the anatomical structure recognition and labeling module 130, and the ultrasound image grading module 140. Moreover, in some embodiments, each of the ultrasound image recognition module 120, the anatomical structure recognition and labeling module 130, and the ultrasound image grading module 140 may be implemented as separate machine learning models, and in other embodiments, some or all of the ultrasound image recognition module 120, the anatomical structure recognition and labeling module 130, and the ultrasound image grading module 140 may be implemented in a same machine learning model.

The machine learning circuitry 105 may be trained based on training images 210. Training images 210 may include any ultrasound image information. For example, the training images 210 may include image information used to train the ultrasound image recognition module 120, such as a variety of ultrasound image information associated with known views of an organ, such as the heart. As a further example, the training images 210 may be clinically desirable images of, e.g., suprasternal views of a heart. In such a case, the training images 210 may be ultrasound images which have been pre-determined (e.g., by a physician) as adequately showing a clinically desirable suprasternal view of a heart. Each such training image 210 may have slightly different characteristics (e.g., higher quality images, lower quality images, blurry images, images taken at slightly different angles, and so on), yet each such training image 210 may nonetheless be pre-determined as adequately representing a clinically desirable view of a heart or other anatomical structure.

Moreover, the training images 210 may include not only image information associated with clinically standard or desirable views, but may further include image information associated with non-clinically standard or desirable views. Accordingly, the ultrasound recognition module 120 may receive, for example, a view of a heart which is not representative of any particular clinically desirable view (e.g., suprasternal, subcostal, short- and long-axis parasternal, 2-chamber apical, 3-chamber apical, 4-chamber apical and 5-chamber apical views). In such a case, the ultrasound recognition module 120 may nonetheless be trained to recognize the image as being a view of a heart, and may further recognize the image as being an image somewhere between, for example, a 2-chamber apical view and a 3-chamber apical view. A clinically standard 3-chamber apical view is generally obtainable, for example, by rotating an ultrasound imaging probe about 60° counterclockwise with respect to the 2-chamber apical view. Ultrasound images obtained with the probe at an angle of rotation somewhere between, for example, 5° and 55° counterclockwise with respect to the 2-chamber apical view may be determined as not representing a clinically desirable view of a heart. The ultrasound image recognition module 120 may be trained with training images 210 showing a variety of known, but non-clinically desirable, views of a heart (such as views somewhere between the 2-chamber apical and the 3-chamber apical views), and thus may recognize such views (e.g., the ultrasound image recognition module 120 may recognize a view as representing a 35° counterclockwise rotation of the probe 118 with respect to the 2-chamber apical view). In some embodiments, upon recognizing an ultrasound image as containing a known non-clinically desirable view, guidance may be provided to the user to move the ultrasound probe in a manner that ultimately achieves acquisition of a clinically desirable view.

In some embodiments, the training images 210 may include image information used to train the anatomical structure recognition and labeling module 130. For example, the training images 210 may include a variety of ultrasound image information associated with known anatomical structures, such as particular organs (e.g., the heart) or particular features of organs (e.g., left ventricle, right ventricle, left atrium, right atrium, mitral valve, tricuspid valve, aortic valve, etc.). Further, the training images 210 may include image information associated with such known anatomical structures from a variety of different views. Anatomic structures may appear very different across different views, e.g., the left ventricle may appear different in ultrasound images acquired at various different views (e.g., apical-LV, parasternal long-LV, parasternal long-LV). Therefore, ultrasound images representing known anatomic structures (e.g., the left ventricle) in a variety of different views may be provided as training images 210, which may be utilized to train the anatomical structure recognition and labeling module 130 to recognize not only the anatomical structure but also the particular view provided by the ultrasound image.

In some embodiments, the training images 210 may include image information used to train the ultrasound image grading module 140. For example, the training images 210 may include a variety of ultrasound images of different image qualities (e.g., higher quality images, lower quality images, blurry images, and so on). The qualities of the training images 210 used to train the ultrasound image grading module 140 may be graded, for example, by an expert such as a physician or other clinician. The qualities of the training images 210 may be graded based on any grading system. In some embodiments, the qualities of the training images 210 may be graded based on a standard grading system, such as the American College of Emergency Physicians (ACEP) grading rubric provided at Table 1 below. Each of the training images 210 may be assigned a particular grade (e.g., 1 through 5) by a physician or other clinician, with the assigned grade representing a quality of the training image 210.

TABLE 1
ACEP Image Quality Grading Rubric

| | Scoring Criteria | | | | |
|---|---|---|---|---|---|
| | 1 | 2 | 3 | 4 | 5 |
| ACEP Image | No | Minimally | Minimal | Minimal | Minimal |
| Quality Grading | recognizable | recognizable | criteria met | criteria met | criteria met |
| Rubric | structures, no | structures but | for diagnosis, | for diagnosis, | for diagnosis, |
| | objective data | insufficient | recognizable | all structures | all structures |
| | can be | for diagnosis | structures but | imaged well | imaged with |
| | gathered | | with some | and diagnosis | excellent |
| | | | technical or | easily | image quality |
| | | | other flaws | supported | and diagnosis |
| | | | | | completely |
| | | | | | supported |

Other training input 220 may further be provided to the ultrasound image recognition module 120 for training. The other training input 220 may include, for example, manually-entered input to adjust or otherwise manage the image recognition model developed in the image recognition module 120 through the training process.

Using training images 210, the machine learning circuitry 105 (including the ultrasound image recognition module 120, the anatomical structure recognition and labeling module 130, and the ultrasound image grading module 140) may implement an iterative training process. Training may be based on a wide variety of learning rules or training algorithms. For example, the learning rules may include one or more of the following: back-propagation, real-time recurrent learning, pattern-by-pattern learning, supervised learning, interpolation, weighted sum, reinforced learning, temporal difference learning, unsupervised learning, and/or recording learning.

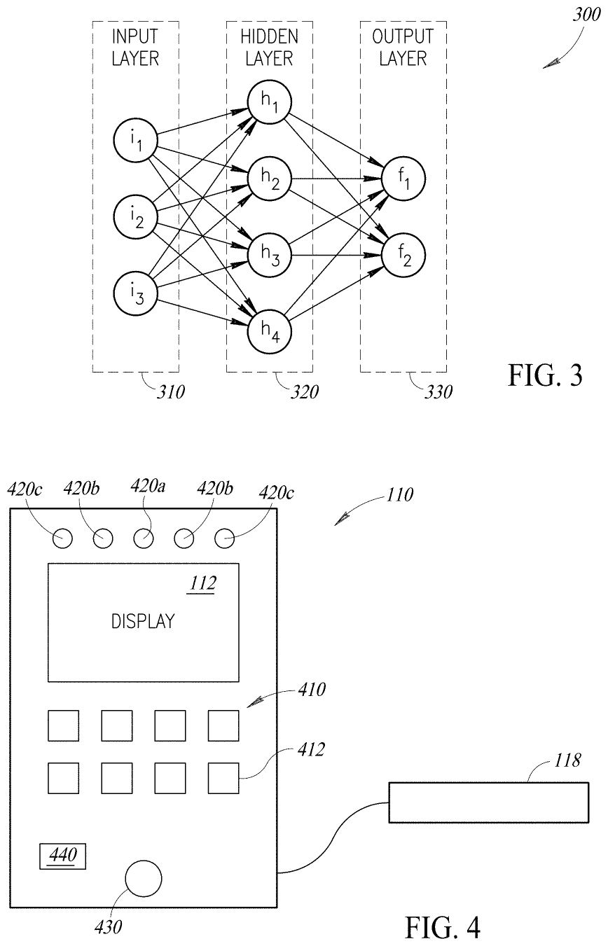

The back-propagation learning algorithm is an example of a method of training artificial neural networks which may be employed, for example, with the artificial neural network 300 shown in FIG. 3. Back-propagation generally includes two phases: propagation and weight update. In the propagation phase, a training pattern's input is forward propagated through the neural network in order to generate the propagation's output activations. Then, the propagation's output activations are backward propagated through the neural network using the training pattern target in order to generate deltas (i.e., the difference between the input and output values) of all output and hidden neurons. In the weight update phase, for each weight-synapse the following steps are generally performed: 1. Multiply its output delta and input activation to get the gradient of the weight; 2. Subtract a ratio (percentage) of the gradient from the weight. The propagation and weight update phases are repeated as desired until performance of the network is satisfactory.

As a result of the training, the ultrasound image recognition module 120, the anatomical structure recognition and labeling module 130, and the ultrasound image grading module 140 may learn to modify their behavior in response to the training images 210, and obtain or generate ultrasound image knowledge 230. The ultrasound image knowledge 230 may represent any information upon which the ultrasound image recognition module 120, the anatomical structure recognition and labeling module 130, and the ultrasound image grading module 140 may determine an appropriate response to new data or situations. For example, the ultrasound image knowledge 230 may represent relationships between ultrasound images and one or more views of an organ (e.g., one or more functions that describe one or more views of an organ based on ultrasound image parameters, coefficients, weighting information, parameters associated with the example neural network shown in FIG. 3 or any such variable), which may be utilized by the ultrasound image recognition module 120 to recognize ultrasound images. Further, the ultrasound image knowledge 230 may represent relationships between received ultrasound image information from a variety of different views and recognized anatomical structures represented in the received ultrasound image information. Additionally, the ultrasound image knowledge 230 may represent relationships between received ultrasound image information and image quality of the received ultrasound image information.

The ultrasound image knowledge 230 may be stored in the ultrasound image knowledge database 122.

Based on the training images 210, the ultrasound image recognition module 120, the anatomical structure recognition and labeling module 130, and the ultrasound image grading module 140 may learn to modify their behavior, and may apply knowledge contained in the image knowledge database 122 to alter the manner in which these modules make determinations with respect to new input, such as, for example, ultrasound image information received from the ultrasound imaging device 110.

FIG. 3 is a block diagram illustrating one example of an artificial neural network 300, which may be implemented by the machine learning circuitry 105, in accordance with one or more embodiments. In some embodiments, each of the ultrasound image recognition module 120, the anatomical structure recognition and labeling module 130, and the ultrasound image grading module 140 may be implemented by a neural network, such as the neural network 300 shown in FIG. 3. Artificial neural networks (ANNs) are artificial intelligence models that are used to estimate or approximate functions that can depend on a large number of inputs, and which are generally unknown. Such neural networks generally include a system of interconnected “neurons” which exchange information between each other. The connections have numeric weights that can be tuned based on experience, and thus neural networks are adaptive to inputs and are capable of learning.

The artificial neural network 300 shown in FIG. 3 includes three layers: an input layer 310 including input neurons i₁ through i₃, a hidden layer 320 including hidden layer neurons h₁ through h₄, and an output layer 330 including output neurons f₁ and f₂. While the neural network 300 of FIG. 3 is shown having three layers, it should be readily appreciated that additional layers may be included in the neural network 300 as desired to achieve optimal training and performance of the machine learning circuitry 105. Similarly, the neurons in each layer are shown for exemplary purposes, and it should be readily understood that each layer may include more, even significantly more, neurons than shown in FIG. 3.

The neural network 300 may be trained by providing training images 210 to the input layer 310. As described with respect to FIG. 2, the training images may include ultrasound image information having a wide variety of known characteristics, including, for example, various organ views, various known anatomical structures at various different imaging views, various image qualities or grades, and so on. Through training, the neural network 300 may generate and/or modify the hidden layer 320, which represents weighted connections mapping the training images 210 provided at the input layer 310 to known output information at the output layer 330 (e.g., classification of an image as a particular imaging view of a heart, recognition of a particular anatomical structure in an image, classification of an image as having a particular image quality). Relationships between neurons of the input layer 310, hidden layer 320 and output layer 330, formed through the training process and which may include weight connection relationships, are generally referred to herein as “ultrasound image knowledge,” and may be stored, for example, in the ultrasound image knowledge database 122.

Once the neural network 300 has been sufficiently trained, the neural network 300 may be provided with non-training ultrasound images at the input layer 310 (i.e., ultrasound images taken of a patient utilizing the ultrasound imaging device 110). Utilizing ultrasound image knowledge stored in the ultrasound image knowledge database 122 (which may include, for example, weighted connection information between neurons of the neural network 300), the neural network 300 may make determinations about the received ultrasound image information at the output layer 330. For example, the neural network 300 may determine whether the received ultrasound images represent one or more clinically desirable or non-clinically desirable views of an organ, and may further recognize one or more anatomical structures in the received ultrasound images and may automatically label the recognized anatomical structures in the images, and still further may automatically determine and assign an image quality grade to the received ultrasound images.

The neural network 300 of FIG. 3 is provided as just one example, among various possible implementations of the machine learning circuitry 105 which employs artificial intelligence to make determinations with respect to received ultrasound image information. For example, the machine learning circuitry 105 (including one or more of the ultrasound image recognition module 120, the anatomical structure recognition and labeling module 130, and the ultrasound image grading module 140) may implement any of neural network, deep learning, convolutional neural network, and Bayesian program learning techniques to make determinations with respect to received ultrasound images of a patient.

Moreover, the ultrasound recognition module 120 may be trained, utilizing a variety of training images 210 and/or a variety of sequences of training images 210, to make a variety of determinations relating to received ultrasound image information. For example, the ultrasound recognition module 120 may be trained or otherwise configured to determine whether a received ultrasound image represents one or more clinically standard or desirable views. Further, the ultrasound recognition module 120 may determine whether a received ultrasound image represents a non-clinically desirable view (and may recognize such non-clinically desirable view as a particular view or angle of a particular organ or other tissue within a patient), and may further determine based on a sequence of received ultrasound images whether the images are approaching or moving away from a clinically desirable view of an organ. For example, if the received ultrasound images are improving in quality from one image to another (e.g., based on the quality assessments provided by the ultrasound image grading module 140), the ultrasound recognition module 120 may determine that the user is moving toward obtaining a clinically desired view of the organ or other anatomical structure. Based on its recognition of whether the images are approaching or moving away from a clinically desirable view of the organ, and/or on its recognition of the actual image captured, the system may then be configured to provide feedback to the user to assist the user in capturing the desired view of the organ, for example, by indicating a direction in which the user may wish to move the probe and/or an angle of rotation or orientation in which the user may wish to angle the probe.

For example, as discussed above, the ultrasound image recognition module 120 may be trained with training images 210 showing a variety of known, but non-clinically desirable, views of a heart (such as views somewhere between the 2-chamber apical and the 3-chamber apical views), and thus may recognize such views (e.g., the ultrasound image recognition module 120 may recognize a view as representing a 35° counterclockwise rotation of the probe 118 with respect to the 2-chamber apical view). Further, the ultrasound image recognition module 120 may be trained with a sequence of recognized, but non-clinically standard or desirable views of a heart. For example, the ultrasound image recognition module 120 may be trained to recognize ultrasound images showing a view of the heart at each degree of counterclockwise rotation between 0° and 60° with respect to the 2-chamber apical view (i.e., every degree between the 2-chamber apical and the 3-chamber apical views). Further, the ultrasound image recognition module 120 may be trained to recognize a sequence of or progression of such non-clinically desirable views toward and/or away from a clinically desirable view (e.g., the training images 210 may include a sequence of ultrasound images representing rotation of the probe 118 from the 2-chamber apical view toward and/or away from the 3-chamber apical view). The ultrasound image recognition module 120 may thus be trained to recognize that received ultrasound images, while not being representative of a particular clinically desired view, may be getting successively closer to (or moving away from) the clinically desired view.

Further, the ultrasound image recognition module 120 may be trained such that the ultrasound image recognition module 120 may determine whether received ultrasound images represent any of a plurality of clinically desirable views of an organ. Such clinically desirable views of an organ may include, for example, suprasternal, subcostal, short- and long-axis parasternal, 2-chamber apical, 3-chamber apical, 4-chamber apical and 5-chamber apical views of a heart.

Referring again to FIG. 1, the machine learning circuitry 105 (including any of the ultrasound image recognition module 120, the anatomical structure recognition and labeling module 130, and the ultrasound image grading module 140) may provide a feedback signal (indicated, for example, by reference numeral 103) to the ultrasound imaging device 110, based on analysis of received ultrasound images by the machine learning circuitry 105, as described in further detail below.

FIG. 4 schematically illustrates an ultrasound imaging device 110, in accordance with one or more embodiments. The ultrasound imaging device 110 may include a display 112, a user interface 410 including one or more input elements 412, one or more visual feedback elements 420, an audible feedback element 430 and/or a haptic feedback element 440.

The user interface 410 allows a user to control or otherwise communicate with the ultrasound imaging device 110. Various types of user input may be provided, for example, via the user input elements 412, which may be buttons or similar user input elements. Additionally or alternatively, the display 112 may be a touchscreen display, and user input may be received via the display 112. Using the ultrasound imaging device 110, a user may select (e.g., via the input elements 412 and/or display 112) or otherwise input a desired view of an organ that is to be imaged in a patient. For example, a user may select one view (e.g., a subcostal view of a heart) from among a plurality of clinically desirable views that are stored in the ultrasound imaging device 110 and presented to the user. The ultrasound imaging device 110 may communicate the selected view to the ultrasound image recognition module 120, and the ultrasound image recognition module 120 may thus be configured to determine whether received ultrasound images represent the selected view. That is, the ultrasound image recognition module 120 may access the appropriate ultrasound image knowledge (e.g., knowledge, rules or relations associated with a subcostal view of a heart) in the image knowledge database 122 such that received ultrasound images may be compared with, or processed by, knowledge corresponding to the selected view. Alternatively, the user may select a mode of operation in which the system guides the user through capture of one of more of a series of standard views of an organ, such as a heart as described above. In such a mode, the system may first select a desired view of the organ to be imaged, and then confirm for the user when the desired image had been captured and/or guide the user towards the desired view based on the initial image capture. For example, when the ultrasound image recognition module 120 determines that a received ultrasound image represents a particular selected view of the organ, the system 100 (e.g., via the ultrasound imaging device 110) may provide an indication to the user (e.g., visual, audible, or haptic feedback) that confirms acquisition of the selected view. On the other hand, if the ultrasound image recognition module 120 determines that a received ultrasound image does not represent the particular selected view of the organ, the system 100 may provide an indication to the user (e.g., visual, audible, or haptic feedback) that guides the user toward acquisition of the selected view, such as by providing an indication of a user motion of the probe 118 in order to acquire the selected view. The indication of the user motion of the probe 118 may include, for example, an indication of a specific direction and/or amount of a rotational or translation motion of the probe 118 in order to acquire the selected view.

The system 100 may then repeat this process, in series, for each of the desired standard views of the organ to be imaged. That is, for each of the series of views of an organ that are desired to be acquired, the system 100 may iteratively guide the user toward acquiring the particular selected view among the series of views, and may confirm when each selected view has been acquired. Alternatively, in some embodiments, the system 100 is configured to operate in such a way to compare any captured image against each of the images to be captured and confirm when one or more of the desired standard views had been captured, without first indicating which view was to be captured first. For example, a particular view does not need to be selected first, in some embodiments. Instead, the system 100 may automatically recognize when a particular desired view (e.g., a clinically standard view of a heart, or the like) has been acquired during an imaging session. Once the particular desired view has been acquired, the system 100 (e.g., via the ultrasound imaging device 110) may automatically provide an indication to the user confirming that the desired view has been acquired, and the user may proceed with the examination of the patient. Similarly, when a second desired view has been acquired, the system 100 may automatically provide an indication the user confirming that the second view has been acquired, and so on. In various embodiments, the system 100 may automatically store the ultrasound images representing the desired views once they have been captured, for example, by storing the ultrasound images in the ultrasound image database 115.

The visual feedback elements 420 may be any element that can provide a visual indication to a user of the ultrasound imaging device 110, and may be, for example, one or more lights, colors, shapes, icons or the like, whether static or moving. The audible feedback element 430 may be any element capable of producing an audible indication to a user of the ultrasound imaging device 110, and may be, for example, a speaker for producing various tones or sounds associated with lack of correspondence and correspondence between the captured image and the image desired to be captured. Similarly, the haptic feedback element 440 may be any element capable of providing a haptic effect to a user of the ultrasound imaging device 110, and may be, for example, a vibration device.

Feedback signals 103 provided by the ultrasound image recognition module 120 may indicate any of a variety of determinations made by the ultrasound image recognition module 120 regarding ultrasound images received from the ultrasound imaging device 110.

For example, the ultrasound image recognition module 120 may provide a feedback signal 103 indicating that a current or most recently received ultrasound image represents a clinically desirable view of the organ (e.g., the selected clinically desirable view). In a further example, the ultrasound image recognition module 120 may determine whether the received ultrasound images are sequentially approaching or moving away from a clinically desirable view of an organ, and provide a feedback signal 103 that indicates whether the received ultrasound images are sequentially approaching or moving away from the clinically desirable view of the organ, e.g., based on increasing or decreasing quality determinations of the ultrasound images by the ultrasound image grading module 140, or based on a sequence of recognized images or structures that are known to be consistent with a progression of images that indicate movement toward or away from a clinically desirable view of the organ. This feedback signal may include a visual or audible command to instruct the user to move or angle the probe in a certain way, or an icon, such as a straight or curved arrow(s), indicating the direction and/or angle of movement required of the probe in order to better approach the desired image of the organ.

The ultrasound imaging device 110 receives the feedback signal 103, and in response, may activate one or more feedback elements (i.e., visual feedback elements 420, audible feedback element 430 and/or haptic feedback element 440) to provide a feedback effect to a user of the ultrasound imaging device 110. For example, the feedback signal 103 may indicate that the current or most recently received ultrasound image represents a clinically desirable view of an organ. In such a case, the feedback effect provided by the ultrasound imaging device 110 may include flashing a green light 420a of the visual feedback element 420, an audible tone or beep from the audible feedback element 430 and/or a vibrational pulse provided by the haptic feedback element 440. The flashing green light 420a, audible tone and/or vibrational pulse indicates to the user that the desired view has been obtained, and the user may thus retain the ultrasound image of the desired view (e.g., utilizing one or more of the user input elements 412) and store the image in an ultrasound image database 115.

Additionally or alternatively, upon determining that a clinically desirable view of an organ is represented in a received ultrasound image, the ultrasound image recognition module 120 may cause (e.g., by a feedback signal 103) the ultrasound imaging device 110 to automatically retain and store the ultrasound image in the ultrasound image database 115. A table may also be displayed with appropriate indications next to each desired type of image, to indicate whether the user had already captured the desired image or whether the desired image remains to be captured for the particular patient being imaged. In some embodiments, the table includes a set of ultrasound images to be obtained in association with a particular type of ultrasound imaging session. The set of ultrasound images may include, for example, a plurality of different views to be acquired during the particular type of ultrasound imaging session (e.g., a cardiac imaging session), such as one or more of: suprasternal, subcostal, short- and long-axis parasternal, 2-chamber apical, 3-chamber apical, 4-chamber apical and 5-chamber apical views of a heart. Various different types of ultrasound imaging sessions may be included within the table, such as ultrasound imaging sessions or examinations for particular pathologies (e.g., lung pathologies, cardiac pathologies, etc.), for particular anatomical structures (e.g., lungs, heart, etc.), or for any other type of ultrasound imaging session or examination. Each of the ultrasound imaging sessions may have an associated set of desired or clinically standard views that should be obtained, and each such view may be included within the table for the particular ultrasound imaging session. Upon acquisition of each of the set of desired or clinically standard views, an entry may be automatically made in the table to indicate acquisition of such view.

In embodiments where a feedback signal 103 indicates that the received ultrasound images are sequentially approaching or moving away from the clinically desirable view of the organ, the ultrasound imaging device 110 may communicate this to the user, for example, by providing a changing feedback effect, such as an audible tone having an increasing (or decreasing) frequency as the received ultrasound images are approaching (or moving away from) the clinically desired view, a series of vibrational pulses having an increasing (or decreasing) intensity as the received ultrasound images are approaching (or moving away from) the clinically desired view, and/or illuminating a different color or position of lights as the received ultrasound image are approaches or moving away from the clinically desired view (e.g., illuminating red outer lights 420c, then yellow intermediate lights 420b, then green center light 420a as the received ultrasound images approach the clinically desired view).

In some embodiments, the feedback signal 103 may represent information derived from or provided by the ultrasound image grading module 140. For example, the feedback signal 103 may indicate a predicted or determined grade of acquired ultrasound images (e.g., a grade of 1 through 5), and the grade may be provided to the user, for example, by displaying the grade along with the displayed ultrasound image on the display 112, or by an audible, visual, haptic, or other feedback mechanism. In some embodiments, the image quality grade, which may be represented by the feedback signal 103, may be utilized to guide the user toward acquisition of a clinically desirable ultrasound image. For example, the feedback signal 103 may indicate that the acquired ultrasound images are of poor or non-clinically useful quality, and the user may thus adjust or reposition the probe 118 until ultrasound images of suitable image quality are obtained.

In some embodiments, the predicted or determined grade of the acquired ultrasound images, which are determined or otherwise confirmed to represent a particular view of an anatomical or structure or organ, may be automatically associated with the particular view and may be stored, for example, in the table.

In some embodiments, the feedback signal 103 may represent information provided by the anatomical structure recognition and labeling module 130 or the ultrasound image grading module 140 in response to analysis of received ultrasound images by the anatomical structure recognition and labeling module 130 or the ultrasound image grading module 140. For example, the anatomical structure recognition and labeling module 130 may recognize an anatomical structure in the received ultrasound images and may automatically provide a label for the recognized anatomical structure, and the label may be displayed along with the ultrasound image, for example, on the display 112.

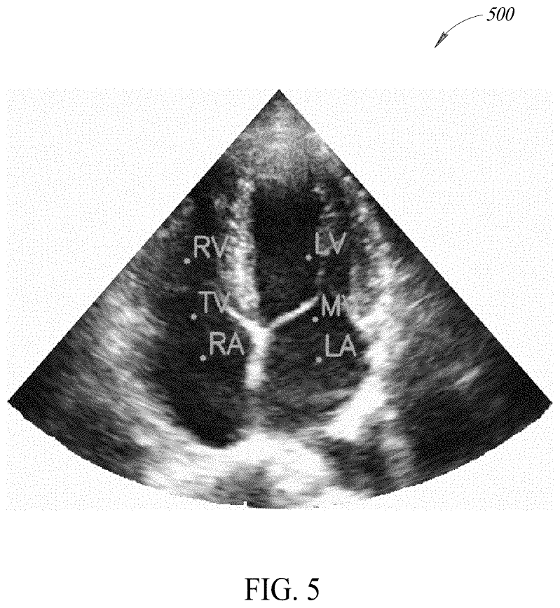

FIG. 5 is a view illustrating an ultrasound image 500 including labels that are automatically associated, by the anatomical structure recognition and labeling module 130, with anatomical structures as recognized by the anatomical structure recognition and labeling module 130. The anatomical structure recognition and labeling module 130 is configured (e.g., through training) to recognize anatomical structures in the received ultrasound images, and to localize the recognized structures, i.e., to recognize or determine the location of the recognized anatomical structures in the ultrasound images. The training of the anatomical structure recognition and labeling module 130 may be performed as described herein, for example, using previously acquired ultrasound images in which anatomical structures have been identified, localized, and labeled in the images, e.g., by human expert interpretation of the ultrasound images. In this way, the anatomical structure recognition and labeling module 130 may determine the correct position for the labels, i.e., at a position corresponding to the determined position of the recognized anatomical structures in the ultrasound images. The labels shown in FIG. 5 include labels for the right ventricle (RV), left ventricle (LV), tricuspid valve (TV), mitral valve (MV), right atrium (RA), and left atrium (LA). These labels are displayed at positions of the displayed ultrasound image corresponding to the structures that the labels identify. The labels shown in FIG. 5 are provided as just some examples of structures which may be automatically recognized and labeled by the anatomical structure recognition and labeling module 130; however, embodiments of the present disclosure are not limited thereto, and in various embodiments labels associated with any anatomical structure may be automatically determined and displayed on the ultrasound images.

As the view in the ultrasound images changes, for example, by movement of the probe 118 and/or the patient being imaged, the labels in the ultrasound image 500 may also change. For example, as recognized anatomical structures appear or disappear from the displayed ultrasound images, the labels corresponding with the recognized anatomical structures similarly appear or disappear. As another example, when anatomical structures in a sequence of displayed ultrasound images are recognized as moving around within the displayed images, the anatomical structure recognition and labeling module 130 may dynamically reposition the labels within the images as the recognized anatomical structures move around within the images.

Moreover, in some embodiments, the outputs of the anatomical structure recognition and labeling module 130 are temporally smoothed in video streams of the acquired ultrasound image data. For example, the results of analysis by the anatomical structure recognition and labeling module 130 (e.g., recognition and labeling of an anatomical structure) may be stored in a circular buffer. In some embodiments, the results (e.g., recognition and labeling of an anatomical structure) that are displayed represent a calculation of the geometric mean of the results in the buffer. In this way, impact of outliers in the determinations being made (presence of and/or position of anatomical structures) in the recognition and labeling of anatomical structures may be diminished, and movement of the displayed labels in the images may be smoothed to reduce jitter or similar effects due to movement of the probe 118, movement of the anatomical structures (e.g., contraction/expansion of the heart), or the like.

In some embodiments, the labels that may be associated with recognized anatomical structures may be restricted based on the view of the acquired ultrasound images. For example, in some embodiments, the anatomical structure recognition and labeling module 130 may recognize not only the anatomical structures represented in acquired ultrasound images, but may further recognize the view at which the ultrasound images are obtained (e.g., apical-LV, parasternal long-LV, parasternal long-LV). As previously described herein, anatomical structures may appear very different in various different ultrasound imaging views. The anatomical structures in various different views may be treated as separate classes for recognition, for example, by the anatomical structure recognition and labeling module 130. The output generated by the anatomical structure recognition and labeling module 130 may thus be restricted in terms of the labels which may be appended or otherwise associated to recognized anatomical structures, depending upon the view of the ultrasound images. In some embodiments, once a particular view of a particular anatomical structure has been determined, the anatomical structure recognition and labeling module 130 may determine labels to associate with the recognized anatomical structure, and the determined labels may be restricted to labels within a particular set of labels which are associated with the particular view of the anatomical structure.

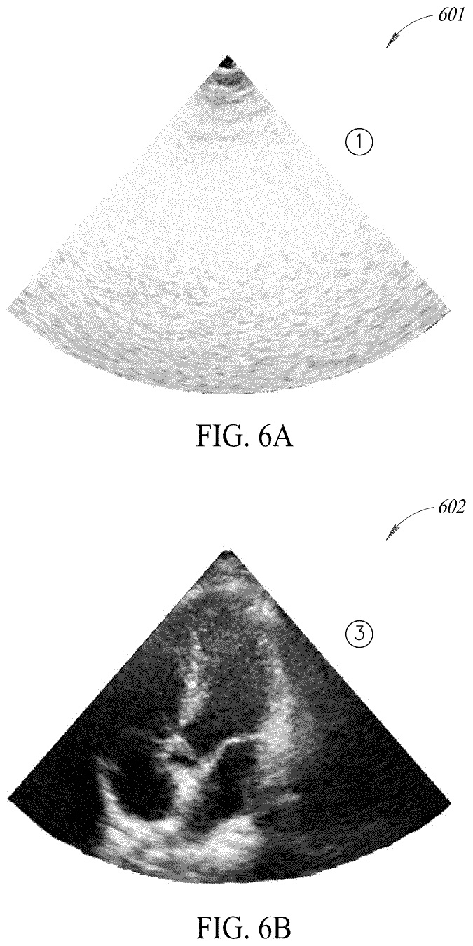

FIGS. 6A and 6B are views illustrating ultrasound images including image grades that are automatically associated, by the ultrasound image grading module 140, with the ultrasound images.

FIG. 6A illustrates an ultrasound image 601 that has been automatically graded as an image quality of “1”, which in some embodiments may represent a low quality image. In some embodiments, as previously discussed herein, a standard grading system such as the ACEP grading rubric (see Table 1) may be utilized or implemented by the ultrasound image grading module 140 in grading the received ultrasound images. The image quality of “1” depicted in the ultrasound image 601 may represent an ultrasound image in which there are no recognizable structures and in which no objective data can be gathered, which is consistent with the standard for a grade or score of 1 in the ACEP image quality grading rubric. In some embodiments, the ultrasound image grading module 140 is configured to annotate the graded ultrasound image with the automatically determined grade. For example, as shown in FIG. 6A, the ultrasound image 601 includes the label “1” which indicates the image grade of the ultrasound image 601 as determined by the ultrasound image grading module 140. Any suitable identifying information may be utilized to display the determined image grade of the ultrasound images, including, for example, numbers, textual description, colors (e.g., red color may indicate poor image quality; green color may indicate good image quality), or the like.

FIG. 6B illustrates an ultrasound image 602 that has been automatically graded as an image quality of “3”, which in some embodiments may represent an intermediate quality image. In some embodiments, as previously discussed herein, a standard grading system such as the ACEP grading rubric (see Table 1) may be utilized or implemented by the ultrasound image grading module 140 in grading the received ultrasound images. The image quality of “3” depicted in the ultrasound image 601 may represent an ultrasound image in which minimal criteria is met for suitability of the ultrasound image for diagnosis and structures are recognizable but with some technical or other flaws, which is consistent with the standard for a grade or score of 3 in the ACEP image quality grading rubric.

In some embodiments, a user may be guided toward acquisition of an ultrasound image of good quality (and in some embodiments, toward acquisition of an image representing a clinically desirable view) through use of the displayed image quality grades. For example, when an ultrasound image is displayed as an image quality of “1”, the user may slowly move or reposition the probe, adjust imaging parameters of the probe (e.g., depth, gain, etc.) or the like. As the user adjusts the probe, the quality of the images may increase, which is used as feedback to the user to indicate that the user is approaching higher quality images.

While the machine learning circuitry 105 (including the ultrasound image recognition module 120, the anatomical structure recognition and labeling module 130, and the ultrasound image grading module 140) has been described herein as being separate from the ultrasound imaging device 110, and accessible via the communications network 102, it should be readily appreciated that the machine learning circuitry 105 may be included within the ultrasound imaging device 110. That is, the machine learning circuitry 105 (including the ultrasound image recognition module 120, the anatomical structure recognition and labeling module 130, and the ultrasound image grading module 140) may be contained within the ultrasound imaging device 110, and may be stored, for example, in memory 114 and the features and/or functionality of the machine learning circuitry 105 may be executed or otherwise implemented by the processor 116.

In some embodiments, one or more of the ultrasound image recognition module 120, the anatomical structure recognition and labeling module 130, and the ultrasound image grading module 140 may be implemented by a single neural network that is optimized for real-time performance on a mobile device. For example, one or more of the ultrasound image recognition module 120, the anatomical structure recognition and labeling module 130, and the ultrasound image grading module 140 may be implemented by a single neural network that is executed by or stored on the ultrasound imaging device 110, and the ultrasound imaging device 110 may be a mobile device such as a laptop or tablet computer, a smart phone, or the like.

In some embodiments, the anatomical structure recognition and labeling module 130 may have an inference time such that anatomical structures are recognized within each one of the received ultrasound images within a time interval such that the processing of the received ultrasound image is complete before the next ultrasound image is available (e.g., is received and available for processing by the anatomical structure recognition and labeling module) during real-time ultrasound imaging. In some embodiments, the anatomical structure recognition and labeling module 130 may be configured to recognize anatomical structures (e.g., object detection), as well as recognize a particular ultrasound imaging view (e.g., view classification), within 25 milliseconds of receipt of acquisition of the ultrasound image information. However, embodiments provided herein are not limited thereto, and in some embodiments, the anatomical structure recognition and labeling module 130 is configured to recognize anatomical structures and ultrasound imaging view in ultrasound images in a time that is less than or greater than 25 milliseconds.

The various embodiments described above can be combined to provide further embodiments. These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. An ultrasound system, comprising:
an ultrasound imaging device configured to acquire ultrasound images of a patient; and
anatomical structure recognition and labeling circuitry configured to:
receive the acquired ultrasound images from the ultrasound imaging device;
automatically recognize one or more anatomical structures in the received ultrasound images; and
automatically label the one or more anatomical structures in the acquired ultrasound images with information that identifies the one or more anatomical structures,
wherein the ultrasound imaging device includes a display configured to display the acquired ultrasound images and the labeled one or more anatomical structures.

2. The ultrasound system of claim 1, wherein the ultrasound imaging device is configured to display the information that identifies the one or more anatomical structures at positions in the displayed ultrasound images which correspond to positions of the one or more anatomical structures.

3. The ultrasound system of claim 1, wherein the anatomical structure recognition and labeling circuitry is further configured to:
automatically recognize a view of the received ultrasound images,
wherein the anatomical structure recognition and labeling circuitry is configured to automatically label the one or more anatomical structures based on a selection of a label from a group of labels associated with the recognized view.

4. The ultrasound system of claim 3, wherein the anatomical structure recognition and labeling circuitry is configured to automatically recognize the one or more anatomical structures in the received ultrasound images, and to automatically recognize the view of each one of the received ultrasound images before a next one of the ultrasound images is received from the ultrasound imaging device.

5. The ultrasound system of claim 1, wherein the anatomical structure recognition and labeling circuitry is further configured to:
determine a plurality of labels for the one or more anatomical structures in each of a plurality of sequentially acquired ultrasound images; and
automatically label the one or more anatomical structures in the images based on an averaging of a determined characteristic of the plurality of labels.

6. The ultrasound system of claim 1, further comprising ultrasound image grading circuitry configured to:
receive the acquired ultrasound images from the ultrasound imaging device; and
automatically grade an image quality of the received ultrasound images.

7. The ultrasound system of claim 6, wherein the display is configured to display an indication of the image quality grade of the received ultrasound images.

8. The ultrasound system of claim 7, wherein the indication of the image quality grade includes at least one of: a number indicating the image quality grade, a textual description indicating the image quality grade, or a color indicating the image quality grade.

9. The ultrasound system of claim 7, wherein the indication of the image quality grade is an integer number from 1 to 5.

10. The ultrasound system of claim 6, wherein the ultrasound image grading circuitry is further configured to:
provide feedback to a user based on the graded image quality, the feedback configured to guide the user toward obtaining a selected view of the one or more anatomical structures.

11. The ultrasound system of claim 6, wherein the ultrasound image grading circuitry is implemented at least partially by machine learning circuitry including at least one artificial neural network.

12. The ultrasound system of claim 1, wherein the anatomical structure recognition and labeling circuitry is implemented at least partially by machine learning circuitry including at least one artificial neural network.

13. A method, comprising:
receiving, by anatomical structure recognition and labeling circuitry, ultrasound images acquired by an ultrasound imaging device;
automatically recognizing, by the anatomical structure recognition and labeling circuitry, one or more anatomical structures in the received ultrasound images;
automatically labeling, by the anatomical structure recognition and labeling circuitry, the one or more anatomical structures in the acquired ultrasound images with information that identifies the one or more anatomical structures; and
displaying the acquired ultrasound images and the labeled one or more anatomical structures.

14. The method of claim 13, wherein the displaying the acquired ultrasound images and the labeled one or more anatomical structures includes displaying the information that identifies the one or more anatomical structures at positions in the displayed ultrasound images which correspond to positions of the one or more anatomical structures.

15. The method of claim 13, further comprising:
automatically recognizing, by the anatomical structure recognition and labeling circuitry, a view of the received ultrasound images,
wherein the automatic labeling includes automatically labeling the one or more anatomical structures based on a selection of a label from a group of labels associated with the recognized view.

16. The method of claim 13, further comprising:
automatically grading, by ultrasound image grading circuitry, an image quality of the received ultrasound images.

17. The method of claim 16, further comprising:
displaying an indication of the image quality grade of the received ultrasound images.

18. An ultrasound system, comprising:
ultrasound image grading circuitry configured to:
receive acquired ultrasound images from an ultrasound imaging device; and
automatically grade an image quality of the received ultrasound images; and
a display configured to concurrently display the acquired ultrasound images and an indication of the image quality grade.

19. The ultrasound system of claim 18, wherein the indication of the image quality grade is an integer number from 1 to 5.

20. The ultrasound system of claim 18, wherein the ultrasound image grading circuitry is further configured to:
provide feedback to a user of the ultrasound imaging device based on the graded image quality, the feedback configured to guide the user toward obtaining a selected view of the one or more anatomical structures.
